(19) United States
(12) Patent Application Publication
(10) Pub. No.: US 2015/0089590 A1
(43) Pub. Date: Mar. 26, 2015

(54) METHODS FOR SECURE CONTROL OF AND SECURE DATA EXTRACTION FROM IMPLANTABLE MEDICAL DEVICES USING SMARTPHONES OR OTHER MOBILE DEVICES

(71) Applicants: Ramnarayan Krishnan, (US); Eugene B. John, Austin, TX (US); Manoj M. Panday, San Antonio, TX (US); Jiwan Limbu Ninglekhu, San Antonio, TX (US)

(72) Inventors: Ramnarayan Krishnan, (US); Eugene B. John, Austin, TX (US); Manoj M. Panday, San Antonio, TX (US); Jiwan Limbu Ninglekhu, San Antonio, TX (US)

(21) Appl. No.: 14/493,305

(22) Filed: Sep. 22, 2014

Related U.S. Application Data
(63) Continuation of application No. 61/880,655, filed on Sep. 20, 2013.

(51) Int. Cl. H04L 29/06; A61B 5/024; G06F 19/00; A61N 1/372

(52) U.S. Cl. CPC H04L 63/10; A61N 1/37235; A61N 1/37252; A61B 5/024; G06F 19/3406
USPC 726/3; 607/59; 600/515

(57) ABSTRACT

The system presented allows secure control of and secure data extraction from implantable medical devices (IMD) using smartphones or other mobile devices. In particular, a patient's or a healthcare provider's mobile device can be utilized to securely interface with an IMD that has been implanted in or on a patient's body. Embodiments include, but are not limited to devices, systems, and methods for securing implanted medical devices.

### BACKGROUND

The present invention generally relates to a system and method of use directed to a system for secure data transmission. More specifically, the present invention relates to a system and method of use for securing transmissions between implantable medical devices using a smartphone or other mobile device.

Information security in the field of health care has become an increasingly complex and important topic in recent years. There has been a thorough investigation regarding threats and vulnerabilities to patients with implantable medical devices, and new design solutions have been described. Information security in the field of healthcare has gained a new urgency and need, and there has been tremendous development in healthcare-related communication networking and information security.

### SUMMARY

Implantable medical devices (IMDs) are smart battery-powered devices with complex circuitry that are able to provide diagnostic information and therapeutic applications to patients with a variety of medical problems. The large majority of IMDs are cardiac rhythm management devices (CRMDs). These include permanent pacemakers and implantable cardioverter-defibrillators (ICDs), which are small battery-powered IMDs that are implanted in patients to treat abnormal heart rhythms called arrhythmias. More specifically, permanent pacemakers are used to treat slow heart rhythms called bradyarrhythmias while ICDs also treat fast heart rhythms called tachyarrhythmias that can cause sudden cardiac death. These devices are interrogated and programmed for diagnostic and therapeutic purposes using a device called an external programmer (EP). An additional example of a CRMD is an implantable loop recorder that provides diagnostic recordings of abnormal heart rhythms. Recent advances in technology have enabled direct communication with CRMDs using a wireless interface. This has improved efficiency and capabilities in the patient care setting. However, wireless technology leaves IMDs vulnerable to security breaches and attacks. This can have important implications for patient privacy and safety.

Certain embodiments are directed to a smartphone or other mobile device-based security scheme for permanent pacemakers and Implantable Cardioverter-Defibrillators (ICDs). Smartphone or other mobile device leverages the Guardian [2] and the Shield [7]. They are external wearable devices that manage communication between the IMD and the external programmer to provide security in regular conditions and safety in emergency situations. External programmers are usually operated by an authorized medical practitioner or a doctor. The described security protocol for access control is based on symmetric key cryptography and uses Smartphone or other mobile device as an authentication and key management device. The external programmers are categorized into unknown and preregistered programmers to entitle them as part of the security system. Preregistered programmers are programmers that have secret key pre-shared with the Smartphone or other mobile device and unknown external programmers are the ones without pre-shared secret key. Preregistered programmers are expected to be available in the patient's regular hospital. Unknown programmers are either used when the patient's ICD is brought across the EP for the first time, somewhere other than the patient's regular hospital, or in an emergency situation where there is no preregistered programmer available. The hospital or clinic other than patient's regular hospital is defined as an away-hospital and unknown external programmers (EPs) are sometimes called Foreign External Programmers.

In certain aspects, an emergency situation is handled in a unique way. As mentioned earlier, human values are important, and the patient is included in the system design. If the patient is conscious and has an ability to respond, this protocol has a feature in which a patient can respond or enter some secret code as input into the Smartphone (or other mobile device) and the External Programmer. This way, the two devices can exchange messages encrypted by the key based on that secret code provided by the patient permanently or for that particular session. A patient also has a right to allow a doctor to input the session secret. If the Smartphone or other mobile device is not participating an authorized medical practitioner can enter a one-time master secret provided by the patient that is shared with the ICD. Policy on how to get a master secret can be further discussed, but as [6] suggests, master secret code can be encoded in a bracelet that identifies the patient as a heart patient or by the policy of the administration. Dimitrios [13] suggests that pre-shared biometric key from the patient be used in case of an emergency scenario. This application presents a scenario where Smartphone or other mobile device is not participating and the patient is incapacitated. In such a scenario, a biometric can be utilized as a secret and unique source for a key. If it is a secret other than biometric, it is suggested that the information may be updated once used.

Smartphones or other mobile devices are very close to desktop computers in computation capability while their flexibility is incredible. Besides being a telephone, it has many different features, including, but not limited to multiple sensors, great audio-visual system, Global Positioning System (GPS), and powerful processor. The computing capability, audio-visual system for security, and real-time monitoring functions of Smartphone or other mobile device are used in various aspects of the currently described device and/or system.

In certain aspects the system and/or related methods use symmetric key cryptosystems in a distinctive way. Symmetric key crypto is free, powerful and consumes less power as compared to public key cryptosystem [13], [15]. Several features of symmetric key cryptography can be used to authenticate an external programmer and secure exchanged messages. Several scenarios and corresponding configurations are described to address possible situations where the devices, systems, or methods can provide a solution to security problems by involving a human as a part of the authentication process.

A system that demonstrates secure communications for all possible threats or attacks, consumes least amount of power as possible, and can fail-open during emergency situation is needed. Smartphones or other mobile devices incorporating security schemes described herein can contribute one or more of the following:

1. Use of a Smartphone or other mobile device as an authentication proxy that protects permanent pacemakers and Implantable Cardioverter-Defibrillator (ICD) against malicious intruders and attackers.

2. Enables registered and unknown programmers to be used to program patient-ICD.

3. Utilizes symmetric key cryptosystem in a smart way to protect ICD from adversaries.

4. Involves the patient as a responder and an authenticator. The use of a Smartphone or other mobile device keeps the patient informed as to what is happening and allows the granting or denial of communication with an external device.

5. Presents Smartphone or other mobile device as a real time monitoring device that keeps the patient informed of, for example, irregular heart activity and displays a secret code in an emergency for use by a medical practitioner.

6. Addresses fail-open secure access for emergency conditions.

7. Utilizes software tools to implement design is and analyze the described security approach.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 illustrates an example message flow between ICD, smartphone and external programmer.

FIG. 2 illustrates registration and message exchange between new EP and the patient's smartphone.

FIG. 3 illustrates message exchange in a preregistered EP.

FIG. 4 illustrates message exchange between EP and ICD in absence of smartphone.

FIG. 5 diagrams a smartphone displaying a temporary secret code.

FIG. 6 diagrams a smartphone screen displaying (a) Authorization dialog box (b) Option to enter the secret code in smartphone.

FIG. 7 diagrams messages displayed on the smartphone indicating the exchange of messages.

FIG. 8 diagrams message exchange activity in EP.

FIG. 9 is a screenshot showing the communication activities on the ICD.

FIG. 10 is a screenshot from smartphone screen simulation of irregular heart activity and secret code receipt.

### DESCRIPTION

Permanent pacemakers and Implantable Cardioverter-Defibrillators (ICDs) are small battery-powered Implantable Medical Devices (IMDs) that are placed in patients' chests to treat abnormal heart rhythms called arrhythmias. More specifically, permanent pacemakers treat slow heart rhythms called bradyarrhythmias while ICDs also treat fast heart rhythms called tachyarrhythmias that can cause sudden cardiac arrest. These devices are programmed and accessed for diagnosis and therapy wirelessly by a device called an External Programmer (EP). Previous studies have shown that permanent pacemakers and Implantable Cardiac Defibrillators (ICDs) are vulnerable to wireless attacks. It is vital in the case of ICDs or any IMDs, an attack may not be limited to extracting patient information but the attack can turn lethal. Some efforts have been made to address this problem in the past. One of the challenges with security in implantable medical devices, such as ICDs, is patient safety. While it is crucial that these devices should be secured by all means possible, a medical practitioner should always be able to access them in an emergency situation.

This application describes a computer security protocol by introducing a Smartphone or other mobile device, which acts as a security management “hotspot” between External Programmer (EP) and Implantable Cardiac Defibrillator (ICD), using symmetric key cryptography. The Smartphone or other mobile device will only allow access to registered programmers or allow unregistered ones with the patient's consent. Several scenarios are described where Smartphone or other mobile device plays a central role in access control including the emergency case.

A medical device is defined as “implantable” if it is either partly or totally introduced, surgically or medically, into the human body and is intended to remain in the body after the procedure [9]. Millions of people around the world live with Implantable Medical Devices (IMDs). A recent study has shown improvement in quality of life and increase in length of life among people who have used IMDs. IMDs come in a range of devices and perform a variety of curative functions, such as deep brain implants for treatment of Parkinson, insulin pumps for diabetes treatment, drug infusion, neuromuscular stimulators that help restore sensation and cardiac pacemakers for patients with rhythm disorders. The devices are mostly electronic and are capable of sensing, computing, transmitting and receiving data over a network, and can be reprogrammed to adjust therapy settings. IMDs usually use short-range telemetry to communicate wirelessly from inside the human body to external equipment because it is undesirable to perform surgery for data retrieval or therapy modifications [9]. The communication specific to these medical devices are known as Medical Device Radio Communications Service (MedRadio). MedRadio spectrum is used for diagnostic and therapeutic purposes in implanted radio devices as well as devices worn on the body. Although there is a wide range of frequencies used for communication with IMDs, the Federal Communication Commission (FCC), Washington D.C. created the MedRadio in 401 to 406 MHz range in 2009 [11].

The Implantable Cardiac Defibrillator (ICD) is one among many different types of IMDs. ICDs are electronic devices invented to help treat irregular heartbeats called arrhythmias. They can sense a rapid heartbeat and can administer electric shocks or pulses to help control arrhythmias, which can cause sudden cardiac arrest (SCA). The device can record and report the incident to a health professional that extracts the collected information using an external device programmer. These battery powered ICD devices are implanted into the human body, particularly in chest or abdomen. Once implanted, ICDs are expected to remain inside the body for up to ten years. Modern ICDs have pacemaker technology that is programmed by external programmers (also called commercial programmers) and monitored by an external monitoring device, both wirelessly without surgery. In this application, the following terms are used interchangeably: External Programmer (EP), External Device Programmer (EDP), Device Programmer (DP), Commercial Device Programmer (CDP) and Programmer.

More than 3,000,000 people in the world have benefited from this technology and predictions show that there will be more people using similar technology in the future. In order to convey messages between the ICD and the external programmer, wireless communication should be carried out. In certain aspects a magnet wand device can be used in conjunction with an EP. The magnet wand is placed in proximity of an implanted ICD where if wirelessly accesses the device. Advancements in technology have resulted in an EP that contacts the ICD wirelessly using radio frequency without requiring a magnet wand. A patient can also be monitored by a wireless trans-receiver, placed usually by the side of the patient's bed that receives wireless data from the ICD and sends it to the doctor or database via internet connection.

Recently, researchers have found that communication-messages between these devices were in plain text or unencrypted and unauthorized access to the ICD was possible. Researchers were able to model an adversary that simulated the function of an EP and could establish communication with ICD [1]. The main vulnerabilities identified were privacy vulnerabilities, wherein the unauthorized party could observe and read private patient data [1], [5]. More decisive were control vulnerabilities in which an unauthorized person can gain control over the ICD's operation and can, for example, disable therapeutic services. The transmitted messages could be captured and modified, could be partly altered during therapy to spoil the medication or therapy, or the ICD could be engaged in communication, which can drain the battery completely and could halt the therapy, putting the patient's life in danger.

The development and power of the internet have augmented our dependency, particularly for information exchange. External monitoring devices have already been used to collect patient's heart activities overnight and send the information to the doctor via the internet. This obviously opens more room for potential threats of all sorts, including attacks such as denial of service, eavesdropping, hacking of the monitor, and many more.

Many interesting design solutions have been made to tackle this issue. There are several reliable cryptographic tools that can address the solution to these problems. Both symmetric (private) and public key cryptography have been described as solution in the past. In Halperin et al. (2008) [1], the author has used symmetric key to encrypt the messages for confidentiality between ICD and commercial programmer. Xu et al. (2011) [2] suggests that an external wearable device, which acts as a guard, be used as an access control device. In Xu et al. [2], the authors describe the use of a symmetric key for message exchange between the guard and the ICD while public key cryptography is used between the guard and the external programmer. One of the major challenges in securing these communications is power. Since, ICDs are battery powered and implanted permanently to last for as long as ten years, the battery power should be handled in a smart and optimal way. While security is a crucial aspect in a medical device, a challenge in any medical device is safety. More clearly, a trained medicine practitioner should be able to have access to the system with ease even in an emergency situation and any security feature should not prevent such access. Therefore, a system that demonstrates secure communications for all possible threats or attacks, consumes least amount of power as possible, and can fail-open during emergency situation is needed. An emergency can be defined as a situation when the patient wearing ICD is incapacitated and immediate access of the information stored in the ICD is needed.

### I. Methods of Security

A computer security system has the following components: Key Management, Entity and Message Authentication, Data Integrity, and Confidentiality. There is no ideal standard security protocol for all the systems. A protocol should be designed for each system taking several components, such as resources, constraints, and attack scenarios. There are two main categories in cryptography: Public Key Cryptography and Symmetric Key cryptography.

In a Symmetric Key Crypto key cryptosystem, all the parties have same key and the key is known as Secret-Key. Same key is used for encrypting and decrypting a message. For example, a message encrypted by key K1 can only be decrypted by key K1. If any party compromised the secret key, it will lead to a security breach.

A public key system also known as asymmetrical-cipher system has a pair of keys. It uses one key for encrypting and another for decrypting a message. The advantage of this system over symmetric key system is that, there is no need for secret-key exchange. But these key pairs are distributed by a major key distribution center. A main advantage of a private system against a public one is that the private is nearly always less computationally intensive and this makes it desirable for usage in low-power systems [13] [15]. Another policy is: an entity has to pay for public key management. In this application, symmetric key cryptography is implemented.

Research has shown that the use of an external device can help secure the ICD. It is known that ICDs are battery powered, low power devices that are expected to remain inside a patient's body for an extended amount of time after implant. Therefore, using the battery power wisely is a vital part in designing a computing system. Implementing security system requires energy. Xu et al [2] suggest a wearable Guardian, which does the message encryption and decryption using symmetric keys generated from ECG signals, for messages between Implantable Medical Device (IMD) and Guardian, and uses a public key crypto for messages between the Guardian and the External Programmer. Authors in [4] also give us direction towards using an external Cloaker, which behaves as a guardian, to the IMD. In their research they propose using light-weight symmetric key algorithm. In both the aforementioned cases, the external security device is removed in an emergency case to directly contact IMD via programmer, and don't apply any security algorithm. Among countless benefits of using Smartphone or other mobile device for information exchange, it is getting more readily available and becoming cheaper, while their functionality and computation power have attained a whole new level. Smartphone or other mobile device have structure like a computer and are almost as strong in computation. It perfectly suits as security proxy for ICD. It is quite easy to program, versatile and interactive device already in our hands.

A. System Components and Security Configuration

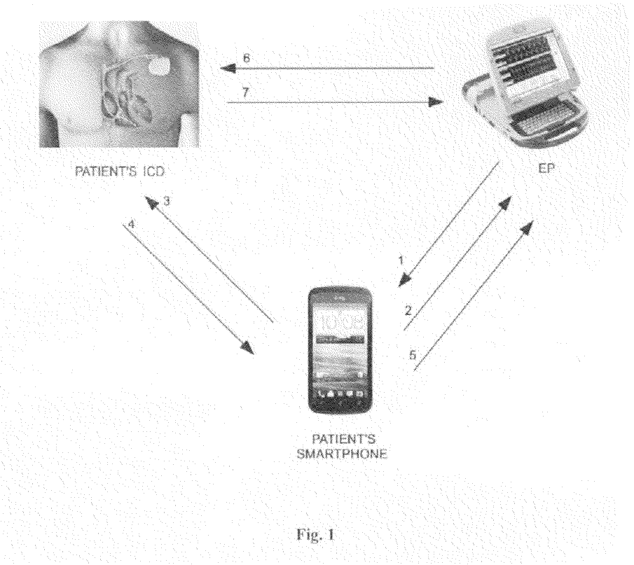

A Smartphone or other mobile device based system consists of four components: the ICD, External Programmer, the patient, and the Smartphone or other mobile device. A simple security configuration among three devices is depicted in FIG. 1.
ICD: An ICD is a device implanted to treat heart condition of a patient. An implantable cardioverter-defibrillator system comprises a pulse generator and one or more leads for pacing and defibrillation electrodes. It contains several components including battery, voltage converters and resistors, capacitors to store charges, microprocessor and integrated circuits to control the analysis of rhythm and the delivery of the therapy. It also has memory chip to store electrographic and other data, and a telemetry module. Once installed, usually remains inside the body for prolonged length of time, sometimes up to ten years [16]. It can communicate with External Programmer via wireless radio up to several meters away [12].
External programmer: Programmer is the device that provides a console for medical practitioner to send data, retrieve data, and change therapy through wireless radio frequency.
The Smartphone or other mobile device: Smartphone or other mobile device is a wireless device such as a wireless phone that has high computation and interactive properties. Smartphone or other mobile device is carried by a human, which in our invented system, acts as an authentication proxy and monitoring module.
The Patient: Patients are themselves an important part of the system in the described security protocol. Research suggests that patients should be informed about any ICD activity so that they can protect or help themselves from the adversaries. Patient acts as authentication stage for any external device.

B. Device Registration and Key Distribution

The central device is the Implantable Cardioverter Defibrillator (ICD). The protocol describes that only one Smartphone or other mobile device is registered with the patient-ICD. At the beginning of the implantation process, a symmetric secret key (K_{ICD} in our example) is exchanged between the Smartphone or other mobile device and the ICD. It is designed such that no Smartphone or other mobile device other than the registered one will be able to communicate with the ICD. At the hospital where the patient had the ICD device implanted, one or more External Programmers (EP) may be available. The external programmers are assumed to be only operated by the authorized medical staffs from the hospital. One or more external programmer can be registered to the Smartphone or other mobile device. That means a Smartphone or other mobile device can share different secret keys with different external programmers or can share a single key with all of them. It is recommended, though, that a unique key be shared with each EP. The programmers with which the Smartphone or other mobile device share the secret key are known as preregistered external programmers (EP). There can be situation where the patient is not located in a regular hospital and is in need of treatment. In that scenario an external programmer without a pre-shared key may be used. Those are categorized as unknown external programmers. Unknown external programmers may be temporarily or permanently registered to the authentication proxy.

In certain aspects it is assumed that the patient-ICD, patient-Smartphone or other mobile device and authorized external programmer follow the protocol as designed. It may also be assumed that the ICD and the external programmer operate within the channel specified by the Medical Implant Communication Service (MICS). In certain aspects the Smartphone or other mobile device is always within the patient's premises and close to the ICD. A Smartphone or other mobile device typically has many functions, including, but not limited to audio and visual capability, computing and communication capability in specified range of frequency, text messaging, and phone and storage capability. It can be worn anywhere around patient's body area or kept in the pocket. It is assumed that Smartphone or other mobile device is patient's private device and only patient can have access to it.

Embodiments described herein relate to wireless attacks, which means that an adversary is assumed to be physically harmless, i.e., the adversary is not able to come and remove or disconnect the phone from the system, or destroy it physically. In certain aspects authorized medical staff or doctor are trustworthy and know how to operate the devices.

C. Adversary Model

One objective is to make the technology most user-friendly, newest ideas are engraved into the product or a system. Every new feature is likely to develop new weaknesses. This application assumes that the adversary's goal is to retrieve data without notice or penetrate patient-ICD in such a way that adversary can modify information inside the ICD. ICD will only respond to the external programmers that are authorized by the Smartphone or other mobile device. An adversary is assumed to be located somewhere around the patient and will attack wirelessly. The attack is assumed to be possible at any time, such as, public places like restaurants, trains, buses etc.

In certain aspects there are two classes of adversaries: Passive Adversary that could violate confidentially of the transmission and Active Adversary that could have access to the patient-ICD or patient-Smartphone or other mobile device and modify the data. They are discussed in more detail as follows:

Passive Adversary.

A passive adversary is someone who is unknown to the user. This adversary listens or captures the transmitted conversation between the patient-ICD and the External Programmer. This adversary is also popularly known as Eavesdropper. The extracted information can be exploited in countless ways against the subject. Passive adversaries may try to decode the transmissions and gain access to confidential information or private information.

Active Adversary.

An active adversary is someone who sends unauthorized wireless command to the patient-ICD. The adversary has an intention to either extract information from the patient-ICD memory or modify the data and controls inside it. It is more hazardous because it can modify patient's data or therapy that can cause physical harm to the patient and even death. If it is able to get inside the ICD, it can also change the ICD configuration in such a way, making ICD to transmit unnecessarily signals or communicate uselessly, drying out the battery.

Active Adversary can attack ICD in the form of Man-in-the-Middle Attack. In this form of attack, the adversary just sits between the ICD and External Programmer and captures the messages from both of them, and can send the message to patient-ICD acting as External Programmer and Vice Versa.

An active adversary may try Replay Attacks. It can record messages from prior communication from other sources and play them back with an expectation of replay from the patient-ICD. This form of attack is called Replay Attacks. It may alter the authorized messages on the authorized channel. Session keys are used in this application whenever possible to mitigate any possibility of replay attacks. It can be assumed that the adversary might transmit using a programmer acquired from market or can simulate a device as an external programmer. In certain aspects the attacks may occur by transmitting higher power signal to the patient-ICD that may cause capture effect as in [3].

In [1], the authors experimentally proved device vulnerability by presenting software based radio (SBR) attacks on privacy, integrity and availability. This literature is among the first ones that actually explained that the information exchanged during communication between Implantable Cardiac Defibrillator (ICD) and External Programmer (EP) is in clear text or unencrypted. Furthermore, they proved that a battery-powered ICD can be made to communicate indefinitely with any unauthenticated device and the device can change the information in the ICD including therapy settings such as shock levels or make ICD issue a commanded electrical shock. Needless to say that also posed risk towards denial-of-service (DoS) attack. Furthermore, the paper presents a defense mechanism of unauthorized attacks, which the author refer to as Zero Power deterrence and prevention system because it doesn't require primary battery power, rather it harvests RF energy from external source. There are three Zero Power components. Zero-power authentication uses symmetric cryptography technique for authentication, zero-power notification audibly warns a patient of security-sensitive actions, and Sensible Security combines elements of zero power notification and authentication that allows the patient to sense the key exchange.

One of the big challenges in securing medical devices is patient safety. In case of emergency, a trained medical assistant should be able to have access without struggle. Denning et al. in [2], [3] give us new research direction in design and implementation of security technique in Implantable Medical Devices (IMDs). The paper explains about using a defensive technique called Communication Cloakers. The author defines communication cloakers as externally worn devices, much like computational medical alert bracelets. It protects the IMD from unauthorized access but allows fail open access during emergencies. The messages are encrypted using symmetric key cryptography. It is possible to fail open during the emergencies just by removing the cloaker and directly contacting the IMD by the Programmer. However, the messages are transmitted and received in plaintext during emergencies. The author further emphasizes the design strategy rather than direct implementation of a cloaker. It suggests that safety should always be the highest concern and fail open should always be considered top priority for patient safety.

Xu et al. in [2] explain technique of using an external device which they name it as Guardian or and the scheme as IMDGuard. The IMDGuard is explained as comprehensive security scheme for heart related Implantable Medical Devices or IMDs. Although it is quite unclear how accurate the signals taken at different points of the body of a patient with weak heart (heart of a heart patient) might be, it tends to describe using human Electrocardiogram (ECG) signals from different locations of the body. Signal from the heart area and other from the wrist, where the Guardian is worn are taken for symmetric key. Two different locations extract same ECG signal to create identical keys for IMD and the Guardian. The Guardian has a list of legitimate external device programmers and their corresponding public keys. The Guardian authenticates the programmers with sending a challenge and validating their signature. It also addresses the patient safety by introducing an emergency protocol wherein the IMD itself sends two consecutive nonces in specified time intervals. The programmer picks up theses nonces, calculates hash of the sum and sends back to the IMD. IMD checks the received message for further communication. They have also described a signal jamming mechanism, for the signal from spoof-attackers that try to force the IMD to go to emergency condition.

In [7] and [8], the authors talk about securing IMDs from active and passive adversaries without changing the design with the help of an external Shield. It is claimed that with this approach it would benefit all the existing IMD's and can be implemented in new IMD's preventing the recall of the existing devices. The shield acts as a gateway that relays messages between the IMD and external commercial programmer. It uses physical layer to secure the communication with the IMD and uses a standard cryptographic approach to communicate between the authorized parties. The shield provides confidentiality by continuously listening to the transmissions and jams them so that it is impossible for eavesdropper to decode. It can simultaneously listen to IMD's signal and transmit jamming signal. The channel creates a linear combination of transmitted signals. Jamming signal with a random signal provides a form of one time pad encryption, where only parties that recognize the jamming signal can decrypt the message.

The literature in [13] suggests using an internal co-processor to secure Implantable Medical Device (IMDs). The IMD processor is divided into two modules. The author claims that it is possible to secure IMD by introducing a co-processor that will coexist with the main IMD processor without consuming too much energy. The author has designed a 5-stage-pipeline RISC application specific processor, a compiler and optimized it for security related computation. Using 90-nm ASIC CMOS technology it claims to have maintained only 8% increase in power and 7% increase in area. It provides a detailed investigation in public key and symmetric key cryptography in many existing encryption algorithm and suggests symmetric key cryptographic algorithm be used. It used MISTY1 as its symmetric key algorithm to find the optimized results.

In [9], the author has investigated a wide variety of Implantable Medical Devices (IMDs). Furthermore, it has done detail research in each subject including wired and wireless technology, weak and unencrypted authentication, weak encryption, safety, unencrypted communication, traffic, firmware, electromagnetic interference, social engineering, types of attacker and physical security. It also investigated different security standards and did assessment of cardiac resynchronization therapy devices. It suggests that security policies are as important as security protocols. It also suggests that security researchers should follow planning and precautions, legal precautions, add ethical barriers, safety measures, formal and clear documentation to their research. In the security recommendations, it suggests a second channel notification for attack recognition and security breach notification. It further demands a “Guaranteed Emergency Access” to the IMDs. It further recommends that biometric authentication be used during emergencies, as tattooed secret were regarded as an unpopular idea by many patients. In this research, biometric authentication approach is utilized during emergency case. This application also describes an emergency notification system via second channel for fatal irregular heart activity.

Kramer et al. talk about the adversaries trying to implant malware into the medical devices turning them into “botnet” in the network among many other software drawbacks [6]. As these medical devices are becoming more connected to the internet for software updates and patient monitoring purposes, protecting them from malicious software is critical area for research. It also alerts researcher for being very cautious when designing and developing security software, since past history have shown that most medical device recalls were from software bugs and software failure. Researches in [3], [5] and [4] put light on design challenges and preferences in regard to human values, interests and assurance.

Certain embodiments of methods and devices described herein are designed to involve human authentication and assurance in various aspects.

### II. Smartphone or Other Mobile Device as Security Device

Certain embodiments include patient and patient-Smartphone or other mobile device as access control proxies for ICD. A standard encryption technique is utilized for confidentiality. One feature is that a patient will be aware of any known or unknown device trying to contact patient-ICD and has power to allow or deny the device from continuing the communication. Solution to this security problem is addressed by presenting different scenarios that will simulate the real life possible cases. Although one goal is to secure the patient-ICD from different adversaries, it is equally important that the safety of the patient be addressed in all cases, especially in an emergency. An emergency for a cardiac patient with ICD is when there is an extreme irregular activity in patient's heart and patient can become incapacitated. Patient's Smartphone or other mobile device carried by the patient is the patient-ICD's proxy, for ICD will only recognize one unique Smartphone or other mobile device programmed to act on its behalf. When an ICD is implanted in a patient, the patient is required to visit hospital frequently for clinical consultation. During these visits the patient may receive new medication, change in ICD therapy or other medical and psychological advices. To continue normal session of data exchange, the external programmer should be first registered before performing any read or write to and from the ICD.

The various components of the invention communicate with one another via various communication interfaces. In certain embodiments, communication interface supports wireless communication links, such as infrared (IR), radio frequency (RF), and audio, among others. Examples of RF wireless links include the IEEE 802.xx family, such as WiFi® (IEEE 802.11), 2.4 ghz Wireless Modules, and Bluetooth® (IEEE 802.15.1). In addition to wireless communication links, communication interface may further support mechanically connected communication links, such as galvanically wired connections, sensor interface connections, connections to external antennas, HDMI, USB, network connections, etc., and may accordingly include a physical adapter or receptacle for receiving such connections. Communication interface may transform an instruction received from processor into a signal sent via a communication medium, such as a network link. It is noted that communication interface may be a bidirectional interface, such that responses, such as commands, information, or acknowledgements, may be received.

The programmers in a patient's regular hospital may be registered into the Smartphone or other mobile devices. Several scenarios are presented for illustration purposed. The cases are divided on the basis of a patient's condition. In certain aspects the patient's condition can be a normal medical consultation or a medical emergency. Normal condition may be referred to as a patient visiting a doctor for regular clinical consultation. Even in clinical settings there might be a case where the patient-Smartphone or other mobile device may not be available. Other cases may be where patient can be located in places other than hospital. The patient may be incapacitated or conscious. In certain aspects a doctor or other medical help is on site.

In the first illustration, a security problem is addressed in a normal case, where a patient goes to regular hospital for clinical consultation. If a new EP is encountered it needs to be registered with the patient's Smartphone or other mobile device before performing further actions. A second illustration explains the case where a Smartphone or other mobile device may not be available during consultation. A third illustration is directed to how a Smartphone or other mobile device can assist medics to access patient-ICD when the patient is incapacitated. A fourth illustration will elaborate on the scenario directed to patient safety. Secure communication is possible even when Smartphone or other mobile device is not present and patient is incapacitated.

Scenario 1: Patient in Clinical Consultation.

Patient is required to visit hospital for regular consultation after the ICD has been implanted. It is described that the patient Smartphone or other mobile device be registered to the patient ICD during implantation. This way the patient Smartphone or other mobile device is already acting as proxy as soon the patient walks out of the hospital after the implant. There can be two cases when the patient visits hospital. Patient may encounter either new EP unregistered to ICD or the one that has been registered before. It is unknown in respect to the patient, patient ICD and Smartphone or other mobile device.

New Programmer, Smartphone or other mobile device is Present.—To convey the programming operation of ICD with an EP, the EP must be first registered to the patient Smartphone or other mobile device. This situation can also take place in a case where a patient is visiting a doctor in a different hospital than the regular one. This protocol offers a technique by which a patient can be referred to a hospital or clinic at a different location where the medical practitioner can still use new external programmer (EP). Two versatile options are offered with this noble approach. First, when a new EP is brought and registered with the Smartphone or other mobile device, the registration information can be stored into the Smartphone or other mobile device. Therefore, if same patient-Smartphone or other mobile device and patient came across same EP in future, they are able to recognize each other. Second, when a new EP is brought into the system, the patient can register EP to the Smartphone or other mobile device temporarily. That is, once the programming session of patient-ICD by temporarily registered EP is over, the Smartphone or other mobile device will forget the EP.

A registration process for a new EP is depicted in FIG. 1. It shows how a new external programmer is registered to the Smartphone or other mobile device with human consent. When an unknown EP requests patient-Smartphone or other mobile device for accessing patient ICD, the patient-Smartphone or other mobile device alerts the patient about the unknown device asking for permission to continue process. Since patient is aware of new EP, allows for further communication. Upon noticing about an unknown EP, the patient-Smartphone or other mobile device wants the new EP to be registered before performing further tasks. The described approach provides a way to register an unknown EP by facilitating the patient to provide an alphanumeric code in Smartphone or other mobile device and new EP. This way the two devices will recognize on the basis of the code and can communicate with each other based on the key derived from the secret code entered by the patient. At this point the unknown EP can be regarded as a registered EP. This registration information can be stored into the Smartphone or other mobile device database in order to facilitate communication between ICD and EP in the future.

In FIG. 1, the unknown EP makes a request with its identity, EP-ID. The patient-Smartphone or other mobile device alerts the patient about a device trying to communicate by popping up a message on the Smartphone or other mobile device screen that gives patient the authority to allow or deny the communication. Once it is allowed, the patient-Smartphone or other mobile device looks if it has previous information about this EP. Since there will be no past record of the new EP, patient-Smartphone or other mobile device will create option to register this new EP by generating a dialog box to provide the secret alphanumeric code. At the same time it sends out a message to the EP about the situation. EP will also give an option to enter the secret code. The patient will provide an alphanumeric secret code in both the devices. The secret code is converted into 128-bit key for communicating with this new EP and stored into the Smartphone or other mobile device database. This way, if this EP tries to access patient-Smartphone or other mobile device in the future, the EP will act as a preregistered EP. This is one way to register a new EP after the implant. But an EP may be registered to the dedicated patient-Smartphone or other mobile device before the implant.

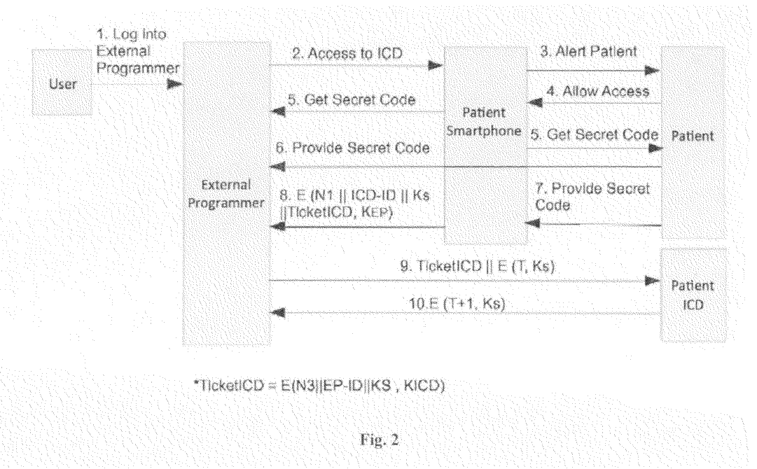

The described protocol also permits the designer with a feature to allow an unknown EP to be registered with the patient-Smartphone or other mobile device temporarily. An unknown EP is registered with the EP for current session only. That is the EP will not be recognized with the same secret code or key in the future. The feature of temporary session registration allows the patient to provide a secret code only for the current session of message exchange. Therefore, the secret is named as session secret code. FIG. 2 depicts how an unknown EP is registered with the patient-Smartphone or other mobile device. This registration can be made a temporary registration by not saving the EP information. FIG. 2 shows the nature of message exchanged when a patient is introduced with unknown EP. This scenario may occur in a new hospital or in a case of emergency where the medical practitioner introduces a new EP. A smart way of using a session secret is described in this scenario. Similar to the first scenario an unknown external programmer attempts to contact patient-ICD and patient-Smartphone or other mobile device is unaware of this new EP.

The patient-Smartphone or other mobile device alerts the patient about this new device trying to communicate with the patient-ICD. Once the patient allows this external device for communication the patient-Smartphone or other mobile device checks if it has any registration information about this device. Since the device is new, patient-Smartphone or other mobile device gives the option to the patient to temporarily register this EP. The patient will be allowed to put same session secret code in both new EP and Smartphone or other mobile device. The hash from the session-secret is derived immediately and used to derive a session key. The session key is saved by both, Smartphone or other mobile device and new EP. At this point the EP is registered. As patient-Smartphone or other mobile device gets the session secret, it generates a random temporary key and sends a token to the EP. The token contains a random nonce, N1, ICD's ID (the ICD Smartphone or other mobile device is trying to reach), a session secret key K_{S}, Ticket to ICD, TicketICD, encrypted with pre-shared secret key, K_{EP}. The EP requests access to the ICD with TicketICD concatenated with a timestamp T, encrypted with session key, K_{S}. TicketICD contains a nonce N₃, EP's ID and session key, K_{S}. All these information is encrypted with the pre-shared key, K_{ICD}. Upon reception, the ICD decrypts the TicketICD and extracts temporary key, K_{S}. The patient-ICD acknowledges back to EP with T+1, encrypted with the session key, K_{S}. Now, the EP and Patient-ICD can communicate with each other.

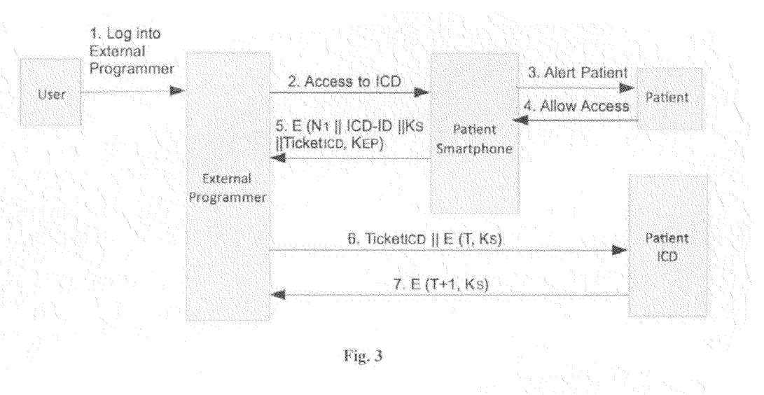

Preregistered External Programmer, Smartphone or other mobile device Present. —Most likely, the patient will visit regular hospital for follow-ups and consultation with dedicated doctors. The patient visits hospital with dedicated patient-Smartphone or other mobile device that is tied to the patient-ICD. Preregistered EP is the EP that already has a pre-shared key with the patient-Smartphone or other mobile device. As mentioned, secret key, K_{EP} is exchanged between the patient-Smartphone or other mobile device and the preregistered External Programmer and secret key, KICD is shared between the patient-Smartphone or other mobile device and patient-ICD. Only one Smartphone or other mobile device per patient is registered with the patient's ICD. The protocol diagram of how three devices communicate is presented in the FIG. 3.

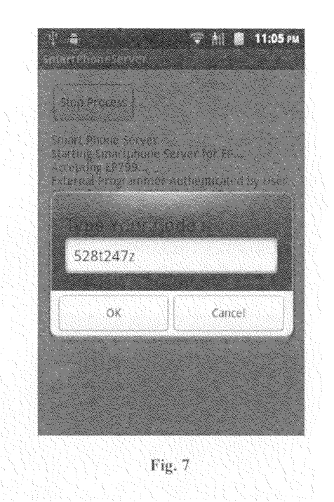

A procedure for programming a patient-ICD can be a scenario wherein a medical practitioner logs in to the preregistered External Programmer (EP) and tries to connect to the patient-ICD. It sends its identity, EP-ID, with request information, encrypted with pre-shared key, K_{EP} to patient-ICD. Since patient-Smartphone or other mobile device has key, K_{EP}, pre-shared, it is able to decrypt and read the message received from EP. As soon as the Smartphone or other mobile device finds out that some external device is trying to connect to the ICD, it displays a message dialog with a Yes/No option, on the Smartphone or other mobile device screen to inform the patient whether the EP should be allowed to communicate further as shown in FIG. 6(a). As the patient knows who is trying to access ICD, patient can allow the EP by pressing ‘Yes’ button. On getting permission from the patient (user), the Smartphone or other mobile device generates a packet for external programmer and sends it to the EP. The packet contains nonce, N1, ICD-ID, and session key K_{S} and Ticket to ICD, TicketICD, encrypted with pre-shared, K_{EP}. EP extracts the received information and sends the TicketICD towards the ICD concatenated with K_{S}-encrypted timestamp, T, with access-request. The TicketICD, as mentioned earlier, contains session key, Ks along with nonce, N3 and EP's ID, that is encrypted with the pre-shared key, KICD.

Upon decryption, the ICD is able to get the session key, K_{S} and acknowledge back by replying with T+1 message encrypted with the extracted key, K_{S}. As shown in FIG. 3, once the EP gets acknowledge back from the ICD, it can them requests information from ICD.

Scenario 2: Clinical Consultation, Smartphone or Other Mobile Device Absent.

Although, it is recommended that Smartphone or other mobile device should be carried by the patient at all times, there might be a situation where the patient-Smartphone or other mobile device might be absent. In such a scenario, the patient-ICD must still be protected. More importantly, at any given situation, an authorized EP must be able to access patient-ICD. This application describes an approach that will maintain the security of patient-ICD even if the patient-Smartphone or other mobile device is not present.

To handle this situation and still maintain security, a key must be pre-shared between patient-ICD and the patient. A secret code called Master Secret code is given to the patient. Master secret code can be printed into the bracelet worn by the patient or patient can choose something he/she can remember. In settings such as clinical consultation, a master secret can also be provided by an authorized doctor from patient's administration record. A key derived from the master secret is kept in the patient-ICD. In a situation where patient-Smartphone or other mobile device is not present, this key will facilitate the EP to communicate with the ICD, with which the patient will share the master secret. This master secret is described to be one-time secret and required to change each time after its usage.

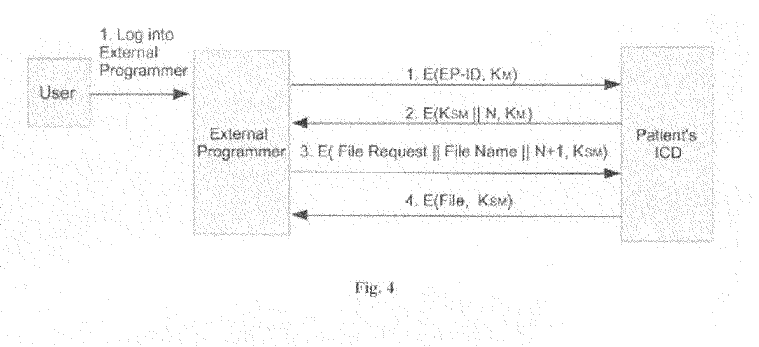

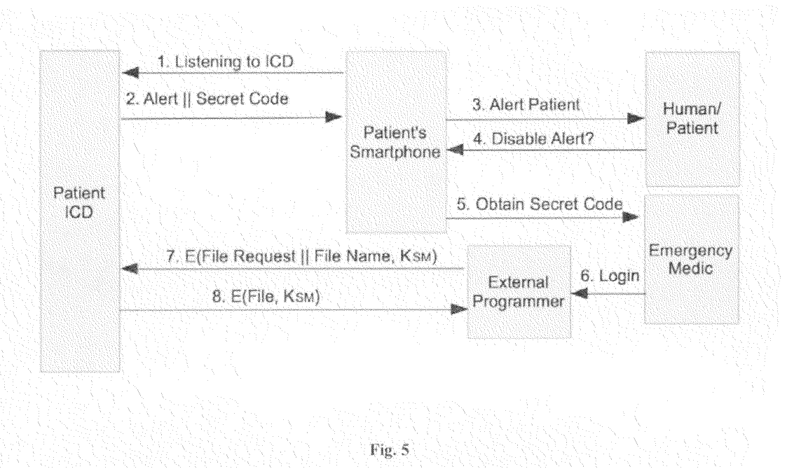

FIG. 4 depicts the block diagram for a situation when the patient visits his/her regular hospital for clinical consultation without Smartphone or other mobile device. The described protocol behaves in following way. EP will have to directly contact the patient-ICD without patient-Smartphone or other mobile device. The doctor enters the master secret code into the External Programmer (EP). The EP generates a key K_{M}, based on the master secret. The EP encrypts the request message with the key generated from the master-secret and sends it to the patient-ICD. The patient-ICD also has the key generated from master-secret stored in it. Thus, it is able to recognize the request. The patient-ICD decrypts the message, identifies the request, generates a random session-secret key, K_{SM} and it sends back this session-secret key concatenated with a nonce, N, to the EP, encrypted with the master-secret key. EP is able to extract the session-secret key and use it to request file from the patient-ICD. In FIG. 4, the EP makes a request of a file by sending a file-request command concatenated with filename and nonce, N+1, encrypted with session key, K_{SM}. A session secret key is a random key generated every time when needed. The patient-ICD delivers the file to the EP encrypted with session key, K_{SM}. This approach can be used with any external programmer.

Scenario 3: Patient Incapacitated, Smartphone or Other Mobile Device Present

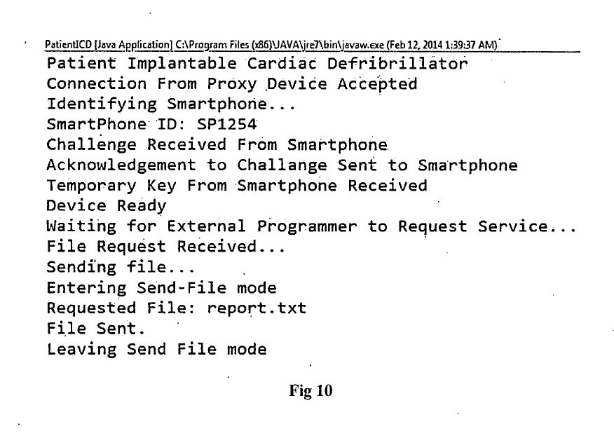

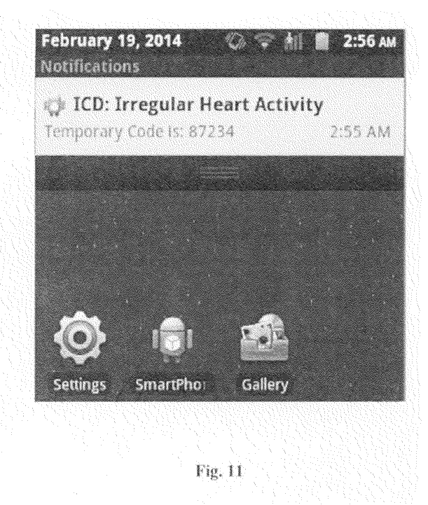

A patient can come across a scenario where he/she becomes incapacitated. This situation can occur anywhere and can be considered as an emergency scenario. This application describes that if the patient is incapacitated and provided that the emergency medical help arrived at the scene on time, the patient-Smartphone or other mobile device can facilitate the emergency medical practitioner with a temporary access code, so that the medical practitioner can access the patient-ICD to get important patient's information. FIG. 10 shows the model of Smartphone or other mobile device as an alert monitoring device and access code facilitator.

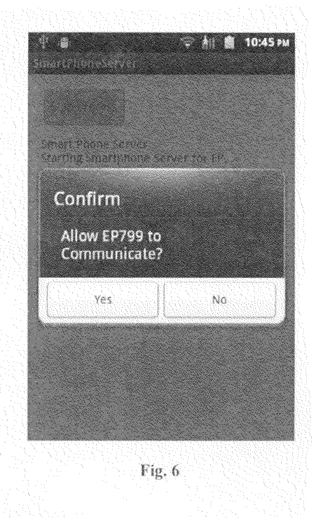

An approach described by this application considers patient-ICD to be continuously monitoring heart activity and patient-Smartphone or other mobile device is continuously listening to the patient-ICD. Patient-ICD is configured to some threshold before it sends an emergency alert message to the Smartphone or other mobile device. If any unusual combination of signals is encountered for instance and considered irregular, then it sends out an emergency alert message with a temporary access code to the Smartphone or other mobile device. Not to mention that patient-Smartphone or other mobile device is listening to the patient-ICD at all times. The ICD sends the message encrypted with the pre-shared key, KICD, as shown in FIG. 5. The alert message pops up on the Smartphone or other mobile device with a sound and vibration. If the patient feels nothing threatening, the patient can discard the message. Provided that the rescue team arrived on time and the patient is incapacitated, the emergency medics can still get the information from the ICD using that key derived from the temporary access code sent to the patient-Smartphone or other mobile device. As shown in FIG. 5, the emergency medical practitioner (a medic) on site obtains the one-time temporary secret from the patient-Smartphone or other mobile device and uses it in the EP to request file directly from the patient-ICD. Request contains request command concatenated with file name requested and encrypted with the temporary key derived from the temporary access code. Again, the key is a temporary, one time secret, which must be updated after the treatment. Since, the key is provided automatically by the patient-ICD, used by the field medics and not patient's doctor, it should have limited access.

Scenario 4: Patient Incapacitated, Smartphone or Other Mobile Device Absent

This scenario is similar to scenario 2 and depicted in FIG. 4, except that the patient is incapacitated. This application provides a method by which a secure communication can be conveyed between EP and patient-ICD in a case where patient-Smartphone or other mobile device is inactive and patient is incapacitated. We assume a scenario in which if an emergency medical help arrives at the scene on time. The medical representative should be able to access the patient-ICD via EP securely without any problem.

For this kind of problem, this application describes that patient biometric information such as fingerprints be used as a key source. Biometrics is unique for each person and therefore extremely difficult to be forged by adversaries. Key derived using biometric information of the patient is shared with patient-ICD during implantation or by programming after implantation. This application also assumes EP with biometric scanner and capability to convert biometrics into a standard digital key. FIG. 4 shows that an EP requests for accessing patient-ICD by using a key, K_{M}, derived from patient's biometric information such as fingerprint via a scanner in an EP (Scanner not shown in the diagram). Since, key K_{M} derived from the biometric information is shared, patient-ICD is able to decrypt the information encrypted with that key. As the ICD gets the initial request, it replies back with session key, K_{SM} along with a nonce, N, encrypted with K_{M}. Session key K_{SM} is used for rest of the message exchange processes. Once EP gets the session key, K_{SM}, it requests the required data from the ICD using, K_{SM}. FIG. 4 shows that EP sends request command concatenated with name of the file and nonce, N+1, encrypted with session key, K_{SM} as a file request. The patient-ICD delivers the file encrypted with K_{SM}.

The following table gives us a quick summary of how the protocol is entitled to behave in different settings:

TABLE 1
Summary of the scenarios addressed by the protocol

| | Patient | Patient State/ | | External | |
|---|---|---|---|---|---|
| Location | Condition | Involvement | Smartphone | Programmer | Remarks |
| Regular | Clinical | Conscious/Yes | Present | New | Registration |
| Hospital | Consultation | | | | Required |
| Regular | Clinical | Conscious/Yes | Present | Pre-registered | Normal |
| Hospital | Consultation | | | | |
| Regular | Clinical | Conscious/Yes | Absent | New/Pre-registered | Master Secret |
| Hospital | Consultation | | | | code from patient |
| | | | | | or doctor |
| Anywhere | Emergency | Incapacitated/No | Present | New/Pre-registered | Temporary |
| | | | | | Access Code |
| Anywhere | Emergency | Incapacitated/No | Absent | New/Pre-registered | Biometrics |

### III. Protocol Implementation and Evaluation

The system basically has four components in the system: patient-ICD, patient-Smartphone or other mobile device, EP and the patient. Simulations for patient-ICD and EP were carried out in Java and Smartphone or other mobile device with Android software. Java has the most portable code based on hardware implementation and since we develop a simple simulation application for an Android Smartphone, the Smartphone or other mobile device is programmed in Android Programming Language. While we implemented the following illustrative example(s) using Java, an Android Smartphone, and Android Programming Language, this invention is not limited to that software and hardware. The invention programming language is not limited to Java, but may include other programming languages such as C and C++. The Smartphone or mobile device and its respective operating system and programming language is not limited to Android but may include Apple iOS, Windows Mobile, Blackberry OS and other mobile device operating systems and its respective programming language and hardware. As explained in previous chapter the protocol can be divided into four major scenarios: Clinical Consultation with Smartphone or other mobile device present, Clinical consultation with Smartphone or other mobile device absent, Patient incapacitated with Smartphone or other mobile device present, and Patient incapacitated with Smartphone or other mobile device absent. Scenarios with incapacitated patient refer safety during emergency scenarios. This chapter will address each scenario with its technical detail as simulated by Java based software and explain how possible threats can be mitigated.

The implementation models the communications among the patient-ICD, the patient-Smartphone or other mobile device and the External Programmer. The data transmission and reception systems of these three devices have many functions in common. There are classes which inherits the function from another class. The implementation consists of three programs that simulate ICD, EP and Smartphone or other mobile device.

ICD: ICD is a monitoring device. The functionality of ICD is represented by six different classes. The classes control the logic of message, file transmission and reception and key generation. A separated class is run as monitoring to simulate monitoring and alert message delivery. Many inbuilt classes from Java are imported as required for encryption-decryption, special coding, different exception handling etc.

Smartphone or other mobile device: Smartphone or other mobile device's functionality is simulated in seven different classes. It has main activity and service activity. Main activity runs and faces the interactive system, display system. Service activity runs in the background which does background functions such as the background processing, listening to the ports, transmitting messages etc. A separate class is created for monitoring the alert message from the patient-ICD. The simulation on this application is done for demonstration purposes only; therefore, decent software development regulations must be followed in production level development of this software.

External Programmer: External Programmer program has six different classes that control main communication and interactive functions. The classes control the logic of messages, file transmission and reception, key generation etc. External programmer does all the functions as ICD and thus contains similar classes. It also uses many inbuilt Java classes.

The wireless communication is simulated by sending and receiving messages using TCP/IP sockets. Each component is assigned static IP address and port number is given to transmit and receives messages. All the transmission and reception was done using Wi-Fi.

The properties of symmetric key cryptosystem can be used in the protocol. Among several symmetric algorithms available Advanced Encryption Standard (AES) algorithm can be used to encrypt messages and files. AES is chained block cipher based on Rijndael cipher. It is the encryption specification established by U.S. National Institute of Standard and Technology (NIST). Among various key sizes available 128 bits key size was used to keep the computation light weight. Even a 128 bit key would take around 1018 years to crack with a brute force attack. On average an attacker will need 1016 years to find the right key with modern super computer [17]. Therefore we can declare 128-bit key as a secure key size. With AES encryption algorithm there is an option to use 192 bits or 256 bits encryption as well. Depending upon the nature of the security system required the encryption strength can be chosen.

The strategy of using secret key also involves using temporary and session keys derived from the secret key rather than the secret key itself. This approach will reduce or prevent the possibility of replay attacks from the adversaries.

Implementation and Evaluation of Described Security Protocol

The operations in the message exchange process involved in both the regular-clinical scenario and emergency scenario are described.

Scenario 1: Clinical Consultation

Unknown Programmer, Smartphone or other mobile device Present.—Any ICD implantation is followed by frequent regular clinical consultations. It is likely that the patient will come across new or an unknown EP. Before any further communication, any new EP should either temporarily or permanently registered with the patient-Smartphone or other mobile device. This scenario also addresses a situation when a patient is referred to an away-hospital. An away-hospital can be referred to as a hospital other than patient's regular hospital, perhaps referred to by the patient's doctor.

Once an Unknown EP tries to access patient-ICD, it sends a request to the patient-Smartphone or other mobile device. The Smartphone or other mobile device alerts the patient about the process by popping up a dialog box that gives an option to the patient to allow or deny further conversation. A patient can deny conversation if an unknown communication is initiated. The patient can allow the EP just by pushing a button in the Smartphone or other mobile device screen. Screenshots in FIG. 6(a) represents the dialog box generated for user consent. After the user allows the EP for further conversation, a new option is displayed in the patient-Smartphone or other mobile device screen. This dialog box allows the patient to enter a secret code and this information is also sent to the EP in same instant. Upon receiving the message from the patient-Smartphone or other mobile device, the EP also gives an option to enter the same secret code into the EP console. The protocol describes that a secret code be used on both the devices and that is shared by entering manually by the patient. As a part of a simulation alphanumeric combination “528t247z” was entered as shown in FIG. 6(b). A 128-bit hash value of the number is derived using MD5 message digest algorithm from which a secret key, K_{S}, is derived using AES standard key generation algorithm. MD5 is 128-bit irreversible hash, i.e. the actual secret cannot be derived from a given hash. Patient-Smartphone or other mobile device and EP are registered at this point. Now, the key derived from the secret code can be stored and used for future processes.

Another useful option is to use an unknown EP temporarily only until the session lasts. In this case, the secret code entered by the patient is called session secret or session secret code, as it lasts only for that session. Patient enters the same session secret in EP and the patient-Smartphone or other mobile device. Again, MD5 hash value of 128-bit is derived from the session secret and converted into AES standard key. The key is then saved into the device's corresponding database. As shown in FIG. 2, as soon as the session key is generated from the secret code, Smartphone or other mobile device sends out temporary key, K_{S} for EP and K_{S} for ICD inside the TicketICD, appended with Smartphone or other mobile device ID, a nonce, N1, encrypted with pre-shared secret key, K_{EP}. The EP sends the TicketICD appended with a challenge timestamp T that is encrypted with, K_{S}. The ICD extracts the key K_{S} from the TicketICD and responds back to the EP with timestamp T+1, encrypted with the temporary session key, K_{S}. The EP can request information from the ICD after this handshake.

The communication between ICD and Smartphone or other mobile device make use of session key, K_{S}. Each time a program is run a new session key is generated randomly using AES encryption algorithm and the session secret key, K_{S} is valid only for limited amount of time.

FIG. 6 (a) shows snapshots of the user's consent dialog box and the session secret user input dialog. (a) Notification dialog for user input (b) User input dialog to enter the session secret

FIG. 6 (a) shows the dialog box which appears in Smartphone or other mobile device screen when an external device tries to access the patient-ICD and sends request towards Smartphone or other mobile device's IP address and port. Patient can choose to allow or deny communication by pressing “Yes” or “No” button. Once the communication is allowed, secret code dialog box appears for user input in the patient-Smartphone or other mobile device and EP. FIG. 6 (b) shows the snapshot of the user input dialog-box. The patient can choose any desired secret code. “528t247z” is shown as an example. Patient can use a new session secret each time the patient-ICD is required to program with new EP. After the temporary key has been delivered in EP and patient-ICD, both the devices are ready to exchange messages. Patient-Smartphone or other mobile device still has the power to stop the file transfer process between EP and patient-ICD. This can be done by pressing the “Stop process” button in the screen as shown in FIG. 7.

This scenario presents great flexibility of using an unknown programmer, equipped with all the security features addressed against the adversaries as explained before. FIG. 7 shows the messages exchanged between the patient-ICD and EP via the patient-Smartphone or other mobile device. The last message sent from the Smartphone or other mobile device is the temporary key for both New EP and the ICD.

As explained earlier, EP also needs the same secret code to continue secure communication with patient-Smartphone or other mobile device. It stops for user input for secret code.

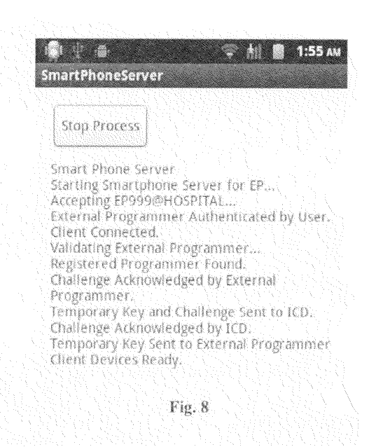

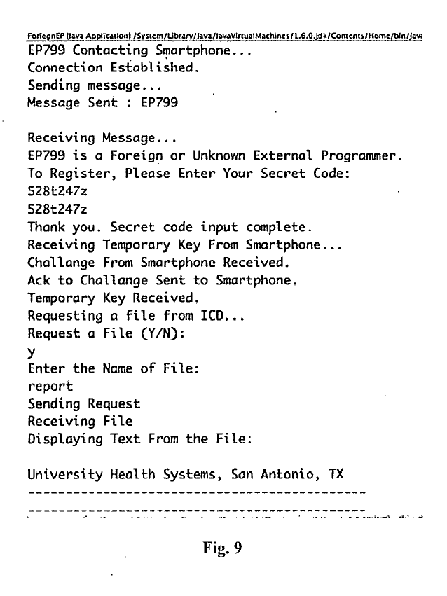

FIG. 8 represents the snapshot taken from the simulated console of a newly introduced EP. It shows that the same session secret was entered into the console of the unknown EP from which hash is generated. This is used to create the session key using 128-bit AES algorithm. The display from the console shows encrypted messages and nature of keys being delivered, represented by symbols and characters. The last two lines from console depicted in FIG. 8 indicate that the file named “report” was requested and could not be received because the request time had expired.

Preregistered EP, Smartphone or other mobile device Present—A regular clinical consultation, wherein a patient is going to regular hospital for clinical consultation, it is most likely that a preregistered programmer will be used. The patient is equipped with Smartphone or other mobile device. With a preregistered EP, a secret key has been exchanged between the EP and patient-Smartphone or other mobile device. Let's say that a preregistered EP is trying to read a file from the ICD. Since, secret key, K_{EP}, is shared between a registered EP and the patient-Smartphone or other mobile device, the EP sends a request encrypted with the secret key, K_{EP} towards the Smartphone or other mobile device's IP address and port number. Smartphone or other mobile device is in a server mode listening for any incoming messages in that specific port. The Smartphone or other mobile device alerts the patient, whether to allow communication with the device by displaying a message in the Smartphone or other mobile device screen as shown in FIG. 6(a). This is where the patient notices an external device is trying to access patient-ICD. Patient can choose by pressing “Yes” or discard the communication request by pressing “No” button on the screen. If patient allows the communication then the rest of the process takes place. Patient-Smartphone or other mobile device is able to decrypt and read the message sent by the EP. Upon knowing that the only purpose of the EP to contact is to get access to the ICD, it sends out a session secret key for EP, K_{S}, to be used for rest of the transmissions. A 128-bit AES standard key is generated randomly. Session key, K_{S} is put into TicketICD. TicketICD dedicated for ICD is concatenated with N1 nonce, target ICD's ID and session key K_{S}, and encrypted with K_{EP}. The EP extracts the TicketICD packet dedicated to ICD, concatenates it with a timestamp, T, encrypts it with the session key, K_{S} and sends it towards ICD. A random generator function from Java library is used to generate a random nonce. The ICD decrypts the TicketICD, extracts K_{S} and replies back with timestamp T+1, encrypted with K_{S}.

When the authentication process is done the EP requests information such as a file to the ICD. On the other end, once the patient-ICD gets the temporary key, it records a time stamp and sets up a specified timer. It waits for the file request in a socket. If the file request message encrypted with the temporary key, K_{S}, is received from an EP within that specifies time period, patient-ICD decrypts the message with temporary key, K_{S}, reads the file request command, locates the requested file and sends out towards the external programmer. If the file request is not received within the specified time, the file request is discarded and the process is broken.

As soon as the communication from the EP is accepted in the first place, the Smartphone or other mobile device application also activates the “Stop Process” button on the Smartphone or other mobile device display. This button allows the user or the patient to stop the file transfer process. This can be useful for the patient to shut the port down as soon as the file transfer is complete or shut it down when required.

The described protocol is able to block both the active and passive adversaries. The secret key is shared only between the parties agreed. Key, K_{EP} is shared only between the registered programmer and the patient-Smartphone or other mobile device, so any adversary requesting access without the key, K_{EP} is discarded by the Smartphone or other mobile device. Moreover, even if the key is compromised and access request is sent to the patient-Smartphone or other mobile device, it displays and notifies about the request to the Smartphone or other mobile device user. If the request is made without the patient or user's consent, the patient can stop the process right there.

The described approach in this application has enforced the idea of using the shared secret key fewer times as possible. It does this by sharing session keys whenever possible. This will limit the exposure of shared secret key to the minimum. This property of key agreement protocol is known as Perfect Forward Secrecy (PFS). This ensures that a session key derived from a set of long-term private or public keys will not be compromised if one of the private keys is compromised in the future [18]. Thus, it is able to protect confidentiality fully. The use of private keys system is free and consumes lot less computation power than public key cryptography [13] [14]. After the temporary key is shared by the Smartphone or other mobile device to request and transfer files between EP and patient-ICD, patient-ICD makes use of a timer that is sets for a specific amount of time. Within which EP has to complete requesting files from patient-ICD.

An additional feature allows patient an authority to stop the file transfer process if required, even after the temporary key, Ks, has been distributed by the Smartphone or other mobile device.

The protocol makes use of nonces, which keeps the transmitted messages fresh. It will reduce the possibility of replay attacks and validates the message between two communicating devices every time. Some of the snapshots from the simulation design program are presented and elaborated in FIG. 8 and FIG. 9.

Eclipse Juno IDE was used for developing the simulation program for External Programmer and the Implanted Cardioverter Defibrillator (ICD). The snapshots for Smartphone or other mobile device display is taken from an emulator by Genymotion that emulated Nexus S Jelly Bean Google Smartphone or other mobile device using Virtual Box as its virtual machine. Android Studio IDE was used to develop the Android application. Huawei Ascend 2 Smartphone or other mobile device with Android Ginger Bread OS were used to demonstrate this simulated application.

Messages displayed in FIG. 7 and FIG. 8 are very similar to the messages when patient-Smartphone or other mobile device is contacted by the preregistered EP. Part (a) of the FIG. 6 shows the notification generated by the Smartphone or other mobile device that asks for patient to decide whether to allow communication with EP. For a preregistered EP, once the consent for communication is given, rest of the process until file request is done automatically.

FIG. 8 displays the snapshot from the console of simulated EP. It lists messages exchanged between itself and patient-Smartphone or other mobile device. For demonstration purposes, we have displayed the encrypted form of messages exchanged, decrypted form of messages when received, the random nonces calculated etc. It also shows the timeline that displays messages when EP contacted Smartphone or other mobile device and upon reception of temporary key, the file requested from the patient-ICD.

Last section of the messages in FIG. 8 indicates human involvement. After the temporary key has been received the EP goes into the file request mode. Once ‘y’ is entered, the EP asks the user for the file name to be requested from the patient-ICD. In FIG. 8, a file named “report” has been requested from patient-ICD, using the temporary key received from patient-Smartphone or other mobile device. In return the file has been delivered and stored in a defined location (not shown here) and the texts from the file are displayed into the console.

FIG. 9 Show the messages displayed in console of the patient ICD. The messages in the console display only shows the important ones. It shows the processes that occurred during clinical consultation, when an EP contacted patient-ICD, after the user and patient-Smartphone or other mobile device authenticated it. It also shows the messages in encrypted form and messages after they were decrypted. Again, these are shown in the given form to exhibit the approach and for simulation reasons. Most of these functions run in the background. As ICD is entitled to establish a timed socket opening, the display shows the time stamp recorded. Once the temporary key is received, the patient-ICD goes into the file-send mode. “Entering Send-File mode” message is displayed in the console.

Scenario 2: Clinical Consultation, any EP, Smartphone or Other Mobile Device Absent

To address the curiosity of “what happens if a patient loses the Smartphone or other mobile device?” or to address the situation when Smartphone or other mobile device is not available, this protocol is described. It makes sure that even without Smartphone or other mobile device the patient-ICD can still stay secured and secure communication can be performed to guarantee patient safety.

Let's assume a circumstance where a patient's Smartphone or other mobile device is not available for any reason during regular consultation with a doctor at hospital. In this case a one-time Master Secret code is used. As mentioned earlier master secret code is a code that is shared with the patient when the ICD is implanted. This code may either be memorized by the patient or imprinted in patient's identity bracelet. Since, this probably will be a clinical consultation with a trusted doctor; doctor can get access to a master secret from administrative sources.

The only way is to make the External Programmer contact ICD is contacting it directly. The master key is just one time and is shared with the patient-ICD. The doctor enters the master secret code. The EP generates the master secret key, K_{M}, using AES algorithm from the MD5 hash of the master secret code, encrypts a request command with the master secret key and sends it to the patient-ICD. Upon reception of the message, patient-ICD replies back a master session secret key, K_{SM}, encrypted with the master secret key, K_{M}. The patient-ICD decrypts the message using, K_{M} and stores master session secret key, K_{SM}, in a file. EP retrieves the key from the file each time it requests file to the patient-ICD. This protocol is used in emergency condition by both registered and unknown programmers.

To reduce the chance of replay attacks, session keys are used whenever possible. Needless to say, authentication and confidentiality are in place, and more importantly, patient safety is also addressed. Integrity in the case of symmetric key cryptosystem can be achieved by calculating HMAC and delivering it to the receiver along with the message. The receiver component can verify the integrity by matching the HMAC calculated from the received message.

Scenario 3: Patient Incapacitated, Smartphone or Other Mobile Device Present

Heart activity monitoring is the key characteristic of an Implantable Cardioverter-Defibrillator (ICD). We describe an extended feature of ICD which makes it capable of transmitting information to patient-Smartphone or other mobile device. In that case patient-Smartphone or other mobile device is continuously listening or monitoring ICD device which plays a key role in delivering important message during emergency. In our simulation, a monitoring process is assumed to be run in the ICD's end, which is simulated by a random number generator. If a random number generator occurs less than 100000, the random number itself is sent to the Smartphone or other mobile device as a temporary secret. As soon as the random number is selected and sent, a time stamp is recoded and timer is started. This timer will represent the temporary nature of that code. The patient-Smartphone or other mobile device on the other end runs a service in android or other mobile device platform which is always listening at a dedicated port for alert messages from the ICD. In reality a patient-ICD is programmed to collect heart activity and certain threshold of signals will initiate it to supply the shock to the heart. This feature can be exploited to little more extent. Any possible life threatening or unusual activity can be sent as alert to the Smartphone or other mobile device. So, as soon as the patient-ICD finds some unusual activity, it sends an alert message. The alert message contains a message and a temporary access code. It is encrypted with the pre-shared secret key, K_{EP} and sent to the Smartphone or other mobile device. The Smartphone or other mobile device displays the alert message with beeping sound and vibration to alarm the patient. A patient can discard the message if he feels okay and reduce the intensity of ongoing activity. Message handling after this point can be designed in a desired way. But, if for some reason the patient falls incapacitated, the message is still on the phone and provided that medical practitioner arrived at the scene on time, they can use the temporary access code from phone into the external programmer. The secret code is input to obtain the hash value, out of which AES based 128-bit key is generated and used as the secret key. This key is time limited and session specific, which will expire.

FIG. 10 shows the simulation of an irregular heart activity. It is simulated by a random number generator. If the random number is under 100000, it is regarded as emergency and first generated random number is sent as a temporary access code. FIG. 10 shows “87234” as random access code sent to the patient-Smartphone or other mobile device. This is received by patient-Smartphone or other mobile device and is displayed as notification with vibration and sound. The message from the ICD appears in the Smartphone or other mobile device activity monitor as shown in FIG. 10. The message reads: “ICD: Irregular Activity, Temp Code: 87234”. The design can provide an option to send SMS or make an emergency call to a desired person once the message in the activity monitor is tapped. Handling the situation after this point can be done in many interesting ways such as sending geographic coordinates with SMS etc.

Described herein are new defensive techniques in improving security, privacy and safety in Implantable Cardioverter-Defibrillators (ICDs) using Smartphone or other mobile devices. The inventors have employed symmetric key crypto in all the communications involved. Although public key cryptosystem has their own advantage, for the described system, the symmetric key cryptosystem addresses the best of its needs. Symmetric keys are cost effective and low power consumers, and hence preferred over public key cryptosystem. The inventors have exploited symmetric key to the best way possible to provide confidentiality and access control in communication. The symmetric keys are shared between the devices and using those keys the communication is secured. Session keys are used whenever possible to improve replay attacks and maintain perfect forward secrecy (PFS). We also used random nonce in messages to validate the received message and keep the transmitted message fresh.

External Programmers (EPs) are divided into two categories: Registered and Unregistered, and addressed the security scenarios accordingly. The described protocol addressed both the active and passive adversaries. The Smartphone or other mobile device is utilized to do most of the computation, key distribution and delivery of notification messages to the user or patient. A patient can play a significant role in protecting himself/herself from the adversaries. One of the unique methodologies used in this application is human involvement. Before beginning any communication, patient is informed about it, and a ‘Go’ command is needed to continue the process.

Different emergency situations can be addressed and handled. Furthermore, the inventors address the problem of fail-open in emergency situations. Finally, the inventors implemented a monitoring functionality in Smartphone or other mobile device that will alert patient about emergency irregular activity in his heart by sending an alert message with a temporary secret-key. This will help medics to access the ICD, if the patient is unconscious, by obtaining the temporary-secret from the phone.

Since Smartphone or other mobile devices have become a part of life for numerous different reasons, carrying it all the time will not distress the patient physiologically. Smartphone or other mobile device is computationally powerful and applicably versatile. Therefore, a lot more feature can be exploited to make the patient life easier and more secure.

The disclosed system and method of use is generally described, with examples incorporated as particular embodiments of the invention and to demonstrate the practice and advantages thereof. It is understood that the examples are given by way of illustration and are not intended to limit the specification or the claims in any manner.

To facilitate the understanding of this invention, a number of terms may be defined below. Terms defined herein have meanings as commonly understood by a person of ordinary skill in the areas relevant to the present invention.

Terms such as “a”, “an”, and “the” are not intended to refer to only a singular entity, but include the general class of which a specific example may be used for illustration. The terminology herein is used to describe specific embodiments of the invention, but their usage does not delimit the disclosed device or method, except as may be outlined in the claims.

Alternative applications of the disclosed system and method of use are directed to resource management of physical and data systems. Consequently, any embodiments comprising a one component or a multi-component system having the structures as herein disclosed with similar function shall fall into the coverage of claims of the present invention and shall lack the novelty and inventive step criteria.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific device and method of use described herein. Such equivalents are considered to be within the scope of this invention and are covered by the claims.

All publications and patent applications mentioned in the specification are indicative of the level of those skilled in the art to which this invention pertains. All publications and patent application are herein incorporated by reference to the same extent as if each individual publication or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

The system and/or methods disclosed and claimed herein can be made and executed without undue experimentation in light of the present disclosure. While the system and methods of this invention have been described in terms of preferred embodiments, it will be apparent to those skilled in the art that variations may be applied to the system and/or methods and in the steps or in the sequence of steps of the method described herein without departing from the concept, spirit, and scope of the invention.

More specifically, it will be apparent that certain components, which are both shape and material related, may be substituted for the components described herein while the same or similar results would be achieved. All such similar substitutes and modifications apparent to those skilled in the art are deemed to be within the spirit, scope, and concept of the invention as defined by the appended claims.

### REFERENCES

[1] D. Halperin, T. Heydt-Benjamin, B. Ransford, S. Clark, B. Defend, W. Morgan, K. Fu, T. Kohno, and W. Maisel. Pacemakers and implantable cardiac defibrillators: Software radio attacks and zero-power defenses. In IEEE Symposium on Security and Privacy, 2008.
[2] F. Xu, Z. Qin, C. C. Tan, B. Wang, and Q. Li. IMDGuard: Securing implantable medical devices with the external wearable guardian. In Proc. IEEE INFOCOM, 2011.
[3] T. Denning, A. Borning, B. Friedman, B. Gill, T. Kohno, and W. Maisel. Patients, Pacemakers, and Implantable Defibrillators: Human Values and Security for Wireless Implantable Medical Devices. In Proceedings of the 28th international conference on Human factors in computing systems. CHI ‘10. Atlanta, Ga., USA: ACM, pages 917-926, 2010.
[4] T. Denning, K. Fu, and T. Kohno. Absence makes the heart grow fonder: New directions for implantable medical device security. In Proc. USENIX Workshop on Hot Topics in Security (HotSec), 2008.
[5] Burleson W., Clark S., Ransford B., Fu. K “Design Challenges for Secure Implantable Medical Devices”, DAC 2012, San Francisco, USA.
[6] Kramer D B, Baker M, Ransford B, Molina-Markham A, Stewart Q, et al. (2012) Security and Privacy Qualities of Medical Devices: An Analysis of FDA Postmarket Surveillance. PLoS ONE 7(7): e40200. doi:10.1371/journal.pone.0040200
[7] Shyamnath Gollakota, Haitham Hassanieh, Benjamin Ransford, Dina Katabi, Kevin Fu They

Can Hear Your Heartbeats: Non-Invasive Security for Implantable Medical Devices
[8] Haitham Al-Hassanieh Application “Encryption on the Air: Non-Invasive Security for Implantable Medical Devices”
[9] On security of Implantable Medical Devices.Master application for obtaining the master of science degree at the TU/e J. Slobbe Mar. 14, 2013
[11] http://www.fcc.gov/encyclopedia/medical-device-radiocommunications-service-medradio
[12] H. Al-Hassanieh. Master Application: Encryption in the air: Non-Invasive Security for

Implantable Medical Devices, MIT, 2011
[13] Siskos Dimitrios, MSc Application “A Co-processor for a Secure Implantable Medical Device”
[14] S Rehman, M. Bilal, B. Ahmad, K. Yahya, A. Ullah, O Ur Rehman. Comparison Based Analysis of Different Cryptographic and Encryption Techniques Using Message Authentication Code (MAC) in Wireless Sensor Networks (WSN), IJCSI International Journal of Computer Science Issues, Vol. 9, Issue 1, No 2, January 2012
[15] M. A L-Rousan, A. Rjoub and A. Baset. A Low-Energy Security Algorithm for Exchanging Information in Wireless Sensor Networks Journal of Information Assurance and Security 4 (2009) 48-59
[16] K. Fu. Inside risks, reducing the risks of implantable medical devices: A prescription to improve security and privacy of pervasive health care. In Communications of the ACM, 2009.
[17] Mark Stamp, Information Security, principle and practice. John Weily & Sons, Inc, Hoboken, N.J.
[18] http://www.onbile.com/info/how-many-people-use-Smartphone or other mobile devices-in-the-world/
[19] J. DiMarco. Implantable Cardioverter-Defibrillators N Engl J Med 2003; 349:1836-47

What is claimed is:

1. A mobile device comprising:
a controller and a memory coupled to the controller, the memory having program instructions stored thereon that, upon execution by the controller, cause the device to receive an access query to a medical device; and
a controller and a memory coupled to the controller, the memory having program instructions stored thereon that, upon execution by the controller, grants or denies access to the medical device.

2. A system where mobile devices may be used by a healthcare provider in order to securely access patient information from an implantable medical device (IMD) for diagnostic purposes or to program an IMD for therapeutic purposes.

3. A system where mobile devices may also be used by patients to securely receive information regarding the status of their IMD or of the status of their own health.

4. A system that would allow healthcare providers to interface with the IMD at the point of care in the outpatient or inpatient settings but still help to avoid unauthorized individuals from doing so in order to prevent loss of private information.

5. Methods to secure communication between mobile devices and implantable medical devices (with external programmers embedded in the mobile device or as an external independent entity) as enclosed herein. The security methods include differentiated access to the information and data stored in implantable medical devices, and differentiated control of operations that can be performed on implantable medical devices, in order to help prevent unauthorized individuals from accessing this private information and from reprogramming the IMD in a way that could potentially result in harm to the patient.

6. A system for securing IMDs using Smartphone or other mobile device as herein disclosed under 4 different scenarios: (1) Patient in clinical consultation, (2) Patient in clinical consultation but smart phone or other mobile device is absent, (3) Patient is incapacitated and smartphone or other mobile device is present, and (4) Patient is incapacitated and smartphone or other mobile device is absent.
